(19) United States
(12) Patent Application Publication
(10) Pub. No.: US 2006/0120974 A1
(43) Pub. Date: Jun. 8, 2006

(54) Nicotine delivery systems

(75) Inventor: David Leslie Mcneight, Cheshire (GB)

Correspondence Address: JACOBSON HOLMAN PLLC, 400 SEVENTH STREET N.W., SUITE 600, WASHINGTON, DC, 20004, US

(73) Assignee: FLUID TECHNOLOGIES LIMITED of GREAT BRITAIN

(21) Appl. No.: 11/340,482

(22) Filed: Jan. 27, 2006

Related U.S. Application Data
(63) Continuation of application No. 09/926,496, filed on Jan. 25, 2002, now abandoned.

(30) Foreign Application Priority Data
May 13, 1999 (GB) 9911037.1

(51) Int. Cl. A61K 9/68; A61K 36/06; A61K 9/50; A61K 9/16; A61K 31/4439

(52) U.S. Cl. 424/48; 424/490; 424/195.16; 514/343

(57) ABSTRACT

A delivery system for nicotine including nicotine encapsulated in a microcapsule system which releases the encapsulated nicotine on contact of the microcapsules with a nicotine solvent.

This is a continuation application of co-pending application U.S. Ser. No. 09/926,496 filed Jan. 25, 2002, which is the nationalization of PCT/GB00/01807 filed May 11, 2000 of which priority is hereby claimed and both are incorporated by reference into the present application in their entireties.

### FIELD OF THE INVENTION

This invention relates to delivery systems for nicotine.

### BACKGROUND OF THE INVENTION

Nicotine is commonly taken in the form of smoking tobacco, in cigarettes, principally, cigars and pipe tobacco. To a lesser extent, tobacco, or a preparation from it, is chewed. More rarely, nowadays, snuff is taken. Smoking is declared to be injurious to health, though nicotine itself, in appropriate quantity, is not harmful in the way smoking is, which is due to components other than the nicotine in cigarette smoke and may even be beneficial—it is reported on numerous occasions as aiding concentration.

Though some question it, nicotine is generally regarded as addictive certainly, increasing taxes on tobacco, Government health warnings and high profile lawsuits brought against tobacco companies by those made terminally ill, or their bereaved, seem to do little to reduce consumption.

There are several products commercially available to help those wishing to quit smoking. These take the form of tablets, chewing gum and patches, all of which are intended to deliver nicotine without the generation of smoke and its associated carcinogenic or otherwise harmful components.

A problem with formulating such products is that nicotine itself is a quite volatile liquid with a boiling point as low as 123°-125° C. at atmospheric pressure, and this makes it difficult to incorporate in products on account of evaporation losses during formulation and the need to seal the products against evaporation of the nicotine for a reasonable shelf life. At the same time, the nicotine must be readily released on use—in the mouth, in the case of gum or lozenge, or through the skin in the case of a patch.

The manner of injection of nicotine is by dissolving in fatty tissue. Nicotine is not readily absorbed in the gut, and no product is intended to be swallowed.

Patches are, of course, somewhat clinical, and while no doubt quite effective, not aesthetically pleasing. Gum is widely regarded as anti-social, often as much so as smoking—there is a disposal problem involved with gum which its users ignore, which has led to its being outlawed in Singapore, a measure which other countries may well follow. Of all the approaches, the most aesthetically acceptable—lozenges, which leave nothing to dispose of and which can be sucked without the sometimes highly objectionable masticating movements—are perhaps the most difficult to formulate, requiring usually elevated temperature processing, leading to nicotine loss through evaporation and an uncertain final dose in the lozenge, and special protection against evaporation from the finished product, if a reasonable shelf life is to be had.

### SUMMARY OF THE INVENTION

The present invention provides a nicotine delivery system that avoids problems of the prior art and which can give rise to improved products across the available range, but particularly in regard to the lozenge.

The invention comprises a delivery system for nicotine comprising nicotine encapsulated in a microcapsule system which releases the encapsulated nicotine on contact of the microcapsules with a nicotine solvent.

The nicotine solvent that may be targeted could be the fatty tissue of the buccal cavity.

The microcapsules may comprise yeast cells. The system may comprise a mixture of cells charged with nicotine and diluent, empty cells.

The system may be presented in a solid carrier from the surface of which microcapsules are gradually released for controlled delivery.

The solid carrier may comprise a saliva-soluble or dispersible substance, and may comprise a lozenge, which may be sugar-based. The lozenge may have such a size, solubility and charge of nicotine that it delivers a dosage of nicotine, in use over a time period between 4 and 20 minutes, equivalent to that delivered by a cigarette. The lozenge may be elongate, between 5 and 20 cm in length and snappable as by having preferential snapping positions into a number of portions each capable of comfortable accommodation in the mouth.

The solid carrier may, however, comprise a chewing gum.

The system may comprise a flavouring substance, which may also be encapsulated in a microcapsule system, and may also comprise a vitamin supplement, which also may be encapsulated in a microcapsule system.

The system may be comprised in a patch.

### BRIEF DESCRIPTION OF THE DRAWINGS

Nicotine delivery systems according to the invention and embodiments of products including the same will now be described with reference to the accompanying drawings, in which:

FIG. 1 is a diagrammatic illustration of a method of preparing microencapsulated nicotine;

FIG. 2 is a view of one embodiment of a lozenge product; and

FIG. 3 is a view of a second embodiment of a lozenge product.

### DETAILED DESCRIPTION AND PREFERRED EMBODIMENTS

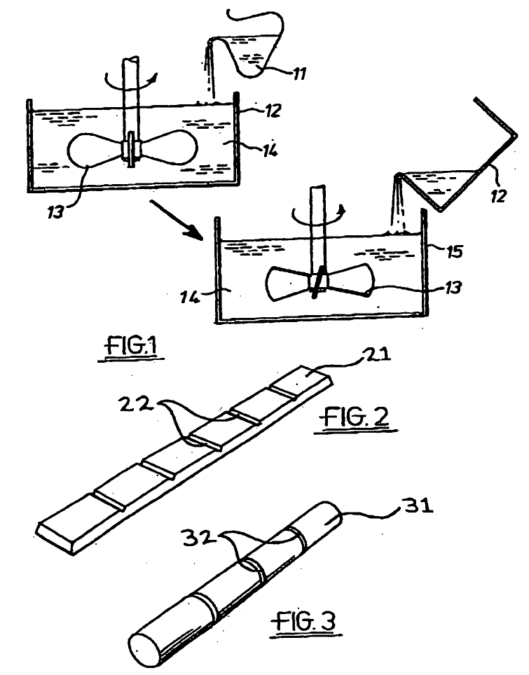

FIG. 1 of the drawings illustrate a method for preparing a delivery system for nicotine comprising nicotine encapsulated in a microcapsule system which releases the encapsulated nicotine on contact of the microcapsules with a nicotine solvent.

Nicotine, in the form of liquid nicotine acid 11, is poured into a mixing vessel 12 with a paddle 13. A measured amount of nicotine is mixed into a given volume of yeast cells 14 in order to give a reasonably concentrated absorption of nicotine into each yeast cell. A suitable mix is 25 g nicotine, 50 g of yeast cells, and 100 g of water. This is stirred for 1-24 hours at about 40° C. Cells are removed by centriftigation and dried. An expected loading is between 25 and 60% by weight of nicotine into the cells, depending on the mix used.

The nicotine loaded yeast cells 14 are then poured from the vessel 12, in a second stage of the process, into a larger volume of yeast cells 14 in a second mixing vessel 15, also with a paddle 13, and the mixture stirred.

Thus, a desired concentration of nicotine encapsulated in yeast cells will be obtained.

The mixed loaded and diluent yeast cells 14 are then incorporated into products with appropriate quantities of the yeast to give the desired nicotine dose in the product.

Two such products are illustrated in FIGS. 2 and 3.

FIG. 2 illustrates an ingot-shaped candy bar 21 which might be some 9 or 10 cm long so as to fit into a packet such as cigarettes are sold in. The bar 21 has transverse grooves 22 enabling it to be snapped into bite-size pieces.

FIG. 3 illustrates a similar product 31, this time shaped more like a cigarette, again with grooves 32 for snapping. The presentations of FIGS. 2 and 3 were first suggested in GB 2 299 756 A.

These products, which are quite similar to cigarettes and which may be used either as aids to quitting smoking or as cigarette substitutes where smoking is not permitted, will, by virtue of their loaded yeast content, contain an equivalent nicotine dose to that delivered by smoking a cigarette.

Flavourings such for example as mint, Scotch whisky, Cognac or menthol can also be added, again encapsulated in similar fashion in the yeast, as can other beneficial agents such as vitamin supplements.

What is claimed is:

1. A delivery system for nicotine comprising:
nicotine encapsulated in a microcapsule system comprising yeast cells, the encapsulated nicotine being released on contact of the microcapsules with the fatty tissue of the buccal cavity.

2. The nicotine delivery system according to claim 1, comprising a mixture of cells charged with nicotine and diluent empty cells.

3. The nicotine delivery system according to claim 1, presented in a solid carrier from the surface of which microcapsules are gradually released for controlled delivery.

4. The system according to claim 3, in which the solid carrier comprises a saliva-soluble or dispersible substance.

5. The system according to claim 4, in which the solid carrier comprises a lozenge.

6. The system according to claim 5, in which the lozenge is sugar-based.

7. The system according to claim 6, having such a size, solubility and charge of nicotine that it delivers, in use over a time period between 4 and 20 minutes, an amount of nicotine equivalent to that delivered by a cigarette.

8. The system according to claim 7, in which the lozenge is elongate, between 5 and 20 cm in length and snappable as by having preferential snapping positions into a number of portions each capable of comfortable accommodation in the mouth.

9. The system according to claim 3, in which the solid carrier comprises a chewing gum.

10. The system according to claim 1, comprising a flavouring substance.

11. The system according to claim 10, in which the flavouring substance is also encapsulated in a microcapsule system.

12. The system according to claim 1, comprising a vitamin supplement.

13. The system according claim 12, in which the vitamin is also encapsulated in a microcapsule system.

14. A delivery system for nicotine comprising yeast cells, the nicotine having a loading of between about 25 and 60% by weight in the yeast cells, the encapsulated nicotine being released on contact of the microcapsules with fatty tissue of the buccal cavity.

15. The nicotine delivery system according to claim 14, further comprising a mixture of cells charged with nicotine and diluent empty cells.

16. The nicotine delivery system according to claim 14, further comprising a solid carrier from the surface of which the microcapsules are gradually released for controlled delivery.

17. The system according to claim 16, wherein the solid carrier comprises a lozenge.

18. The system according to claim 17, wherein the lozenge has a size, solubility and charge of nicotine that delivers, in use over a time period between 4 and 20 minutes, an amount of nicotine equivalent to that delivered by a cigarette.

19. The system according to claim 18, wherein the lozenge is elongate, between 5 and 20 cm in length, and snappable by having snapping positions to form a number of portions each capable of comfortable accommodation in the mouth.
